(19) United States
(12) Patent Application Publication
(10) Pub. No.: US 2005/0107430 A1
(43) Pub. Date: May 19, 2005

(54) Antimicrobial and antiviral compounds

(76) Inventors: Bimal K. Banik, Edinburg, TX (US); Frederick F. Becker, Houston, TX (US)

Correspondence Address: WILLIAMS, MORGAN & AMERSON, P.C., 10333 RICHMOND, SUITE 1100, HOUSTON, TX, 77042, US

(21) Appl. No.: 10/971,882

(22) Filed: Oct. 22, 2004

Related U.S. Application Data
(60) Provisional application No. 60/516,608, filed on Oct. 29, 2003.

(51) Int. Cl. A61K 31/445; A61K 31/135; A61K 31/05

(52) U.S. Cl. 514/319; 514/656; 514/732

(57) ABSTRACT

Disclosed herein are methods of inhibiting infection by at least one microorganism or at least one virus by administering to an animal in an amount effective to inhibit infection a compound having a formula selected from the group consisting of 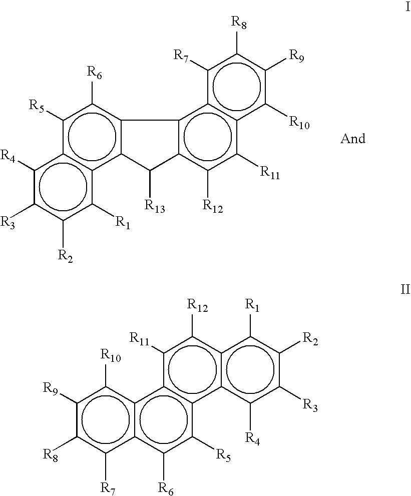
or a salt thereof, such as a hydrochloride salt. At least one of R₁-R₁₃ in formula (I) or at least one of R₁-R₁₂ in formula (II) is —R₁₄Z, where R₁₄ is a substituted or unsubstituted linking group comprising from 1-12 carbon atoms, and Z is a substituted or unsubstituted heterocyclic group having from 1-12 carbon atoms.

This application claims priority from U.S. provisional application 60/516,608, filed on Oct. 29, 2003, which is incorporated herein by reference.

### BACKGROUND OF THE INVENTION

The present invention relates to compounds and methods of administering compounds having antimicrobial and/or antiviral activity to animals (e.g., mammals). Specifically the present invention relates to administering certain chrysene and dibenzofluorene derivatives to animals for the prophylaxis and treatment of infection (e.g., microbial or viral).

When antibiotics were initially identified, they were treated as miracle drugs, and the overuse of these drugs occurred quickly. Certain antibiotics are losing their effectiveness as bacteria evolve resistance to antibiotics that are used to treat bacterial infections. Health officials are concerned about recent outbreaks of drug-resistant bacterial infections in the United States. According to the CDC, 13,300 U.S. hospital patients died of bacterial infections because of antibiotic resistance in 1992 alone. Furthermore, the similarity of many existing antibiotics means that it is possible for bacteria to develop resistance to several antibiotics at once, making infections more difficult to treat.

Tuberculosis is caused by Mycobacterium tuberculosis. The bacterium can attack various organs and parts of the body, but usually attacks the lungs. In the 1940s, scientists discovered the first of several drugs used to treat tuberculosis, and other drugs have been discovered and used since then. As a result, tuberculosis slowly began to disappear in the United States, but tuberculosis has made a come back in recent years. WHO (e.g., The World Health Organization) estimates that eight million people are infected with tuberculosis every year, of whom 95% live in developing countries. An estimated 3 million people die from tuberculosis every year. The recommended four drug regimen of antibiotics that is currently employed in humans consists of isoniazid, rifampin, pyrazinamide and ethambutol or streptomycin. Strains of M. tuberculosis resistant to at least one of streptomycin and pyrazinamide, among other anti-tuberculosis drugs are being encountered.

The ability of certain bacteria (e.g., M. tuberculosis, S. aureus, among others) to develop resistance to antibiotics represents a major challenge in the treatment of infectious disease. Unfortunately, relatively few new antibiotic drugs have reached the market in recent years. Methods for administering new classes of antibiotics might provide a new scientific weapon in the war against bacterial infections.

There are only a handful of antifungal drugs known for the treatment of mammals. In fact, there were only ten FDA approved antifungal drugs available in 2000 for the treatment of systemic fungal infections. There are three important classes of fungal drugs for the treatment of systemic infections: polyenes, pyrimidines, and azoles. The FDA has also approved certain drugs belonging to other classes for topical treatment of fungal infections. Certain traditional antifungal drugs may have a significant toxicity, and certain antifungal drugs available for use in treatment have a limited spectrum of activity. Still further, certain antifungal drugs among the azoles can have interactions with coadministered drugs, which can result in adverse clinical consequences. As with the antibiotics, certain fungi have developed resistance to specific antifungal drugs. Patients with compromised immune systems (e.g., AIDS) patients have in some cases had prolonged exposure to fluconazole for both prophylactic and therapeutic purposes. In 2000, increased use of the drug fluconazole correlated with the isolation of increasing numbers of resistant infectious fungi among AIDS patients. Methods of using a new class of antifungal drugs could make new treatments for fungal infections possible.

Malaria is a serious, often fatal, disease in humans and certain other primates caused by a protozoan parasite (e.g., eukaryotic parasite). There are four kinds of malaria that can infect humans: Plasmodium falciparum, P. vivax, P. ovale, and P. malariae. The World Health Organization estimates that 300-500 million cases of malaria occur and more than 1 million people die of malaria yearly. Malaria occurs in over 100 countries and territories. More than 40% of the people in the world are at risk. The results of resistance studies carried out by MSF-Epicentre in Mbarara, Uganda in 1998 and 2000 showed high levels of resistance to classical antimalarial drugs in the region: —1998: 81.1% resistance to chloroquin and 25% resistance to Fansidar. —2000: resistance levels to Fansidar up to 60%. Thus, resistance is also prevalent in malarial organisms. Methods of using a new class of anti-malarial drugs could make successful treatments for malaria possible.

A number of antiviral treatments are known, particularly for immunocompromised patients. Antiviral compounds can be used to treat (a) infection caused by herpes simplex virus, varicella-zoster virus, human immunodeficiency virus (HIV), cytomegalovirus, or respiratory syncytial virus, (b) viral hepatitis and (c) influenza. Antiviral treatments for HIV have garnered much attention in recent years. Currently there are 18 distinct antiviral drugs used to fight the HIV virus that are approved for use in the USA. These drugs are used in many different combinations to combat HIV infection, and can, in themselves, be toxic to the patient. HIV, as certain other viruses, has proved to be capable of developing resistance to various antiviral compounds. For HIV, it has been found that the development of drug resistance can be reduced by using a combination of drugs, but it can be difficult to identify combinations that are maximally effective that are not overly toxic to the patient. New antiviral drugs that can be used for the prophylaxis and treatment of viral infections are desirable.

### SUMMARY OF THE INVENTION

Certain embodiments of the present invention are directed to methods of inhibiting (e.g., bacteriostatic or bactericidal inhibition for bacteria) the growth of at least one microorganism (e.g., bacteria, fungi, or protist). Such inhibition of growth involves administering to an animal an amount of an antimicrobial drug, as described below, that is effective to inhibit microbial growth. An antimicrobial compound used in methods of the present invention can have a formula selected from the group consisting of 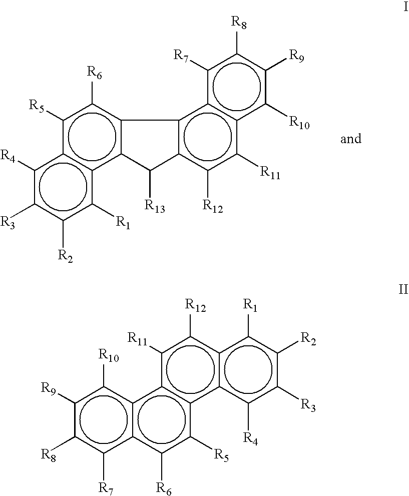
or a salt thereof. At least one of R₁-R₃ in formula (I) is —R₁₄Z, or at least one of R₁-R₁₂ in formula (II) is —R₁₄Z. R₁₄ is a substituted or unsubstituted linking group that comprises from 1-12 carbon atoms. In certain embodiments R₁₄ comprises at least one of an amino or an amido group. Z is a substituted or unsubstituted heterocyclic group having from 1-12 carbon atoms. Preferably, Z is selected from the group consisting of morpholinyl, pyrrolidinyl, piperidinyl and piperazinyl.

In certain embodiments, R₁₄ (of either (I) or (II)) has the formula —NHR₁₅—, where R₁₅ is a substituted or unsubstituted aliphatic group having from 2-6 carbon atoms. Preferably, R₁₅ is selected from the group consisting of —CO(CH₂)ₙCO— and —(CH₂)ₘ— where n is from 1-4, and m is from 2-6.

The remainder of R₁-R₁₃ in formula (I) are independently selected from the group consisting of hydrogen, hydroxyl, halogen, nitro, methoxy, acyl, alkyl groups having from 1-12 carbon atoms, and substituted or unsubstituted pendant groups comprising (i) from 1-12 carbon atoms and (ii) at least one of an amino group or an amido group. Preferably —R₁₄Z in formula (I) is at R₁₁.

The remainder of R₁-R₁₂ in formula (II) are independently selected from the group consisting of hydrogen, nitro, substituted or unsubstituted hydrocarbyl groups having from 1-12 carbon atoms, and substituted or unsubstituted pendant groups comprising (A) from 1-12 carbon atoms and (B) at least one of an amino group or an amido group. Preferably R₂ or R₆ of formula (II) is —R₁₄Z and the remaining positions are hydrogen.

Bacteria for which certain compounds used in methods of the present invention can be bacteriostatic or bactericidal include certain species of Staphylococcus, Stenotrophomonas, Enterococcus, Plasmodium and Mycobacterium. Certain compounds used in the present invention can inhibit fungal growth of certain fungi, such as some Candida. In some embodiments of the present invention certain strains of Staphylococcus aureus (methicillin-resistant, MRSA, and vancomycin resistant, VRSA), Stenotrophomonas maltophilia, vancomycin-resistant Enterococcus faecium (VRE), Mycobacterium fortutium, Mycobacterium tuberculosis, Mycobacterium avium intracellulare, Pseudomonas aeruginosa, and Candida albicans may have their growth inhibited. Certain compounds of the present invention can be used to inhibit growth of certain P. falciparum. Certain microorganisms, which are resistant to at least one antimicrobial known in the art, can have their growth inhibited using methods of the present invention. In certain embodiments, the microorganisms can be resistant to at least one antimicrobial selected from the group consisting of amikacin, ampicillin, amoxicillin/clavulanate, ampicillin/sulbactam, aztreonam, cefazolin, cefepime, cefoxitin, ceftazidime, ceftizoxime, ceftriaxone, ciprofloxacin, clindamycin, erythromycin, gentamicin, imipenem, oxacillin, penicillin, rifampin, tetracycline, tobramycin, trimethoprim/sulfamethoxazole, linezolid, methicillin, vancomycin, imipenem, levofloxacin, meropenem, norfloxacin, piperacillin, piperacillin/tazobactam, ticarcillin/clavulanate, and combinations thereof.

Certain embodiments of the present invention are directed to methods of prophylaxis or treatment of a viral infection in an animal. The methods comprise administering to an animal at least one compound having formula (I), formula (II) or salts thereof, as described above for use in antimicrobial treatments. The compound is administered to the animal in an amount effective to inhibit infection of at least some of the animal's cells by at least one virus.

Certain methods of the present invention may be useful in new antimicrobial and antiviral treatments for patients, alone or in combination with other treatments known in the art.

### BRIEF DESCRIPTION OF THE DRAWINGS

The following drawing forms part of the present specification and is included to further demonstrate certain aspects of the present invention. The invention may be better understood by reference to the drawing in combination with the detailed description of specific embodiments presented herein.

FIG. 1 depicts a scheme for the synthesis of Tx-84, Tx-109, Tx-147 and Tx-225.

FIG. 2 depicts a scheme for the synthesis of Tx-118 and Tx-197.

### DESCRIPTION OF SPECIFIC EMBODIMENTS

The terms “microbes” and “microorganisms” as used herein encompass protists, bacteria and fungi.

In some embodiments, methods of the present invention can involve the use of certain compounds that can inhibit microbial growth in mammals and in humans. In certain embodiments, methods of the present invention can be used for prophylaxis and treatment of viral infection in animals. Methods of the present invention can be applied to certain animals, including certain bovine, porcine, canine, feline, and equine mammals, and avian animals among others. Compounds used in methods of the present invention preferably inhibit growth of pathogenic microbes or inhibit viral infection.

Certain methods of the present invention involve inhibiting the growth of at least one microorganism, comprising the step of administering to an animal in an amount effective to inhibit microbial growth a compound having the formula (A) 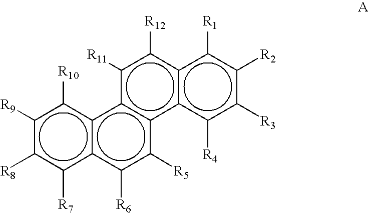
or a salt thereof, such as a hydrochloride salt.

At least one of R₁-R₁₂ of formula A is —R₁₄Z, where R₁₄ is a substituted or unsubstituted linking group having from 1-12 carbon atoms, and Z is a morpholinyl, pyrrolidinyl, piperazinyl or a piperidinyl group. In certain embodiments, R₁₄ further comprises at least one of an amino or an amido group. The remainder of R₁-R₁₂ of formula A are independently selected from the group consisting of hydrogen, nitro, substituted or unsubstituted hydrocarbyl groups having from 1-12 carbon atoms, and substituted or unsubstituted pendant groups having from 1-12 carbon atoms and at least one of an amido group or an amino group. Preferably —R₁₄Z is at position R₂ or R₆ and the remaining positions of the formula are hydrogens.

Preferably R₁₄ of formula A has the formula —NHR₁₅—, where R₁₅ is a substituted or unsubstituted aliphatic group having from 2-6 carbon atoms. Preferably R₁₅ is selected from the group consisting of —CO(CH₂)ₙCO—, and —(CH₂)ₘ—, where n is from 1-4, and m is from 2-6.

Certain embodiments of the present invention are directed to methods of prophylaxis or treatment of a viral infection in an animal. The methods comprise administering to an animal at least one compound having formula (A) or a salt thereof, as described above. The compound is administered to the animal in an amount effective to inhibit infection of at least some of the animal's cells by at least one virus. Viral infection can be inhibited by inhibiting viral replication and/or inhibiting the activation of virus particles that enables the virus particles to attack new cells.

Certain methods of the present invention involving administering a compound having formula (A) can comprise inhibiting the growth of a microorganism. The microorganism can be certain protists, bacteria, or fungi. In certain embodiments, the microorganism can be selected from a genus selected from the group consisting of Staphylococcus, Stenotrophomonas, Enterococcus, Pseudomonas, Mycobacterium, Plasmodium and Candida. The microorganism that has its growth inhibited can be a strain selected from the group of species consisting of Staphylococcus aureus (such as those that are methicillin-resistant, MRSA), Stenotrophomonas maltophilia, vancomycin-resistant Enterococcus faecium (VRE), Mycobacterium fortutium, Mycobacterium tuberculosis, Mycobacterium avium intracellulare, Pseudomonas aeruginosa, Plasmodium falciparum, and Candida albicans.

Certain embodiments of the present invention involving administering a compound having formula (A) can comprise inhibiting viral infection. The infectious virus can be a herpes simplex virus, varicella-zoster virus, human immunodeficiency virus (HIV), cytomegalovirus, respiratory syncytial virus, viral hepatitis or influenza virus.

Preferably the compound having formula (A) is selected from the group consisting of: N-[(6′-chrysenyl)-4-(4′N methyl-piperazinyl)-butane-1,4-diamine]-N-[(12′ chrysenyl)-4-(4′N methyl-piperazinyl)-butane-1,4-diamine hydrochloride [Tx-147]; N-[(6′-chrysenyl)-4-piperidinyl-butane-1,4-diamine]-N-[(12′ chrysenyl)-4-piperidinyl-butane-1,4-diamine [Tx-84]; N-[(6′-chrysenyl)-4-piperidinyl-butane-1,4-diamine]-N-[(12′ chrysenyl)-4-piperidinyl-butane-1,4-diamine hydrochloride [Tx-225];N-(6′-chrysenyl)-4-(4′N methyl-piperazinyl)-butane-1,4-dicarboxiamide [Tx-1]; N-(2′-chrysenyl)-4-(4′N-methyl-piperazinyl)-butane-1,4-dicarboxiamide [Tx-2]; N-(6′-chrysenyl)-4-(1′ piperidinyl)-butane-1,4-dicarboxiamide [Tx-3]; N-(2′-chrysenyl)-4-(1′ piperidinyl)-butane-1,4-dicarboxiamide [Tx-4]; and N-(2′-chrysenyl)4-(1′-piperidinyl)-butane-1,4-diamine [Tx-5]. More preferably the compound is N-[(6′-chrysenyl)-4-(4′N methyl-piperazinyl)-butane-1,4-diamine]-N-](12′ chrysenyl)-4-(4′N methyl-piperazinyl)-butane-1,4-diamine hydrochloride [Tx-147].

In certain embodiments the compound is selected from the group consisting of Tx1-Tx5. This group of compounds is especially well-suited to inhibit growth of Mycobacterium tuberculosis or Mycobacterium avium intracellulare. In some embodiments, the compound is Tx-5, and it is well-suited to inhibit growth of certain Stenotrophomonas maltophilia, vancomycin-resistant Enterococcus faecium (VRE), Candida albicans, and Staphylococcus aureus. In certain embodiments, the compound can be Tx-147, which can inhibit the growth of certain Staphylococcus aureus, Stenotrophomonas maltophilia, vancomycin-resistant Enterococcus faecium (VRE), and Mycobacterium fortutium.

Certain methods of the present invention involve inhibiting the growth of at least one microorganism (as described above) or inhibiting a viral infection (as described above), comprising the step of administering to an animal (as described above)in an amount effective to inhibit microbial growth or inhibit viral infection a compound having the formula (B) 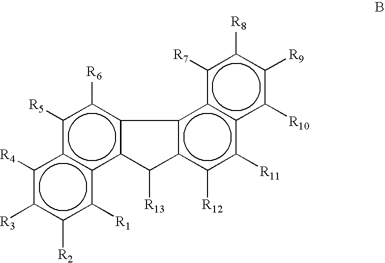
or a salt thereof, such as a hydrochloride salt. R₁-R₁₀, R₁₂, and R₁₃ are independently selected from the group consisting of hydrogen, hydroxyl, halogen, nitro, methoxy, acyl, alkyl groups having from 1-12 carbon atoms, and substituted or unsubstituted chemical groups comprising (i) from 1-12 carbon atoms and (ii) at least one amino or amido group. R₁₁ is —R₁₄Z where R₁₄ has the formula —NHR₁₅—. R₁₅ is a substituted or unsubstituted aliphatic group having from 2-6 carbon atoms. Preferably R₁₅ is selected from the group consisting of —CO(CH₂)ₙCO— and —(CH₂)ₘ— where n is from 1-4, and m is from 2-6. Z is a piperazinyl or a piperidinyl group. Preferably a compound having formula (B) is used to inhibit growth of certain Staphylococcus, such as Staphylococcus aureus, or certain Enterococcus, especially vancomycin-resistant Enterococcus faecium (VRE). Preferably the compound having formula (B) is N-[11′-(13′H-Dibenzo[a,g]-fluorenyl)]-4-(4′N methyl-piperazinyl)-butane-1,4-dicarboxiamide [Tx-37], and in certain embodiments Tx-37 is used to inhibit growth of Staphylococcus aureus, or vancomycin-resistant Enterococcus faecium (VRE).

Certain compounds used in methods of the present invention are administered to an animal (e.g., a mammal) in an amount effective to inhibit the growth of microbes (as described above) or inhibit viral infection in the animal(as described above). The administration can suitably be oral, parenteral and by intravenous, intraarterial, intramuscular, intralymphatic, intraperitoneal, subcutaneous, intrapleural, or intrathecal injection. Such administration is preferred for systemic infections in a patient. In certain embodiments, the administration can be topical (e.g., a salve or a liquid, among others), intraorbital or intracapsular. Such administration is preferably repeated on a timed schedule until infection has essentially been eliminated, and can be used in conjunction with other forms of antimicrobial and/or antiviral therapy.

Certain methods of the present invention can be used with immunocompromised or transplant patients. The compounds of the present invention can also be used to treat surfaces (e.g., countertops and surgical instruments among others), and for the preparation of antimicrobial and/or antiviral reservoirs (e.g., reservoirs in surgical implants and wound dressings).

A compound of the present invention is preferably administered in a dose that is between approximately 0.01 and 100 mg/kg of body weight of the animal (e.g., mammalian) subject. The dose is preferably high enough to have an antimicrobial or antiviral effect, but less than would be toxic to the animal that is being treated.

Certain tests for antimicrobial activity of Tx-1, Tx-2, Tx-3, Tx-4, Tx-5, Tx-9, Tx-10, Tx-11, Tx-12, Tx-37, Tx-38, Tx-84, Tx-109, Tx-112, Tx-I 18, Tx-147, Tx-197, and Tx-225 (see Table 1 below) are described in the present application. Methods for synthesizing some of the compounds are described in U.S. Pat. Nos. 6,362,200, 6,184,224, and 6,015,811, which are incorporated herein by reference.

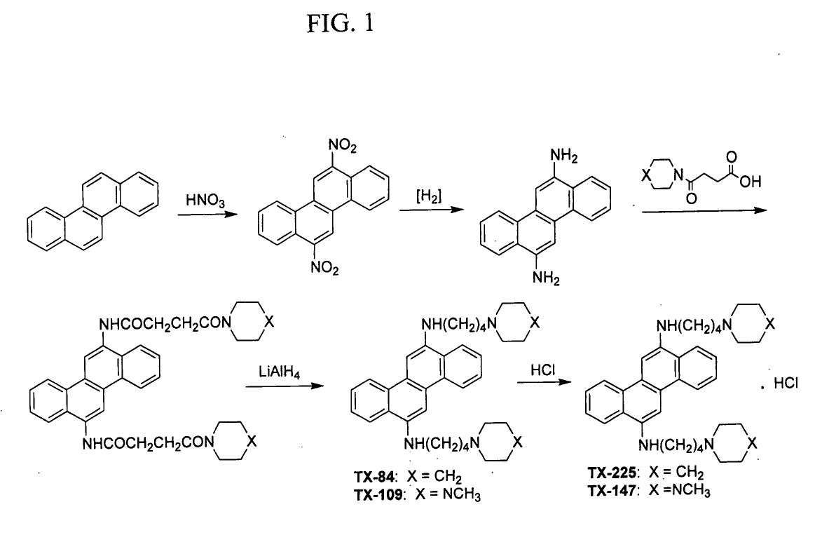

FIG. 1 depicts a scheme for the production of Tx-84, Tx-109, Tx-147, and Tx-225. Chrysene upon nitration under forcing conditions afforded a 6,12-dinitrochrysene. This was reduced by hydrogenation to 6,12-diaminochrysene. A coupling reaction was then performed with the amine and an acid to produce a tetramide. The tetramide was then reduced and the tetramines Tx-84 and Tx-109 were obtained in good yield. Tx-84 and Tx-109 were then converted to the hydrochloride salts Tx-225 and Tx-147.

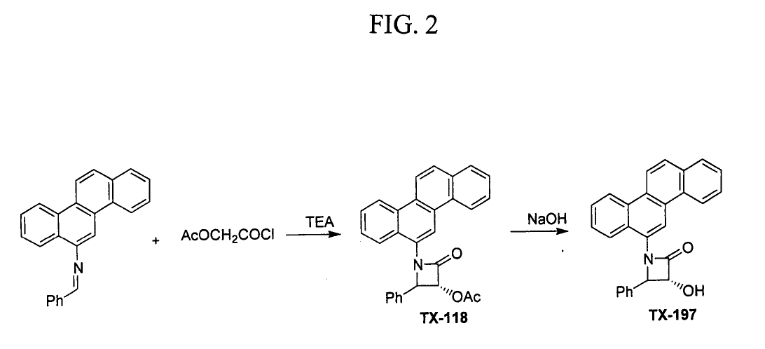

FIG. 2 depicts a scheme for the production of Tx-118 and Tx-197. Cycloaddition of an imine derived from 6-aminochrysene and benzaldehyde with acetoxyacetyl chloride in the presence of triethylamine gave a trans beta-lactam Tx-118. Hydrolysis of Tx-118 was achieved for the preparation of the hydroxyl derivative Tx-197.

TABLE 1

| Compound No. | Compound Name |
|---|---|
| Tx-1 | N-(6′-chrysenyl)-4-(4′N methyl-piperazinyl)-butane-1,4-dicarboxiamide |
| Tx-2 | N-(2′-chrysenyl)-4-(4′N-methyl-piperazinyl)-butane-1,4-dicarboxiamide |
| Tx-3 | N-(6′-chrysenyl)-4-(1′-piperidinyl)-butane-1-4-dicarboxiamide |
| Tx-4 | N-(2′-chrysenyl)-4-(1′-piperidinyl)-butane-1,4-dicarboxiamide |
| Tx-5 | N-(2′-chrysenyl)-4-(1′-piperidinyl)-butane-1,4-diamine |
| Tx-9 | N-(12′-bromo, 6′-chrysenyl)-4-(4′N methyl-piperazinyl)-butane-1,4- |
| | dicarboxiamide |
| Tx-10 | N-(12′-bromo, 2′-chrysenyl)-4-(4′N methyl-piperazinyl)-butane-1,4- |
| | dicarboxiamide |
| Tx-11 | N-(12′-bromo, 6-chrysenyl)-4-(1′-piperidinyl)-butane-1,4-dicarboxiamide |
| Tx-12 | N-(12′-bromo, 2′-chrysenyl)-4-(1′-piperidinyl)-butane-1,4-dicarboxiamide |
| Tx-37 | N-[11′-(13′H-Dibenzo[a,g]-fluorenyl)]-4-(4′N methyl-piperazinyl)- |
| | butane-1,4-dicarboxiamide |
| Tx-38 | N-[11′-(13′H-Dibenzo[a,g]-fluorenyl]-4-(1′-piperidinyl)-butane-1,4- |
| | dicarboxiamide |
| Tx-84 | N-[(6′-chrysenyl)-4-piperidinyl-butane-1,4-diamine]-N-[(12′chrysenyl)- |
| | 4-piperidinyl-butane-1,4-diamine |
| Tx-109 | N-[(6′-chrysenyl)-4-(4′N methyl-piperazinyl)-butane-1,4-diamine]-N- |
| | [(12′chrysenyl)-4-(4′N methyl-piperazinyl)-butane-1,4-diamine |
| Tx-112 | N-{11′(13′H-dibenzo [a,g]-fluorenyl]-4-(1′piperidinyl)-butane-1,4- |
| | diamine |
| Tx-118 | N-(12′-acetyl, 2′-chrysenyl)-4-(4′N methyl-piperazinyl)-butane-1,4- |
| | dicarboxiamide |
| Tx-147 | N-[(6′-chrysenyl)-4-(4′N methyl-piperazinyl)-butane-1,4-diamine]-N- |
| | [(12′chrysenyl)-4-(4′N methyl-piperazinyl)-butane-1,4-diamine |
| | hydrochloride |
| Tx-197 | Trans-1-N-(6′-chrysenyl)-3-hydroxy-4-phenyl-2-azetidinone |
| Tx-225 | N-[(6′chrysenyl)-4-piperidinyl-butane-1,4-diamine]-N-[(12′chrysenyl)-4- |
| | piperidinyl-butane-1,4-daimine hydrochloride. |

The present invention can be further understood from the following examples.

### EXAMPLE 1

Standard NCCLS (e.g., National Committee for Clinical Laboratory Standards) methods for determining MIC (e.g., minimum inhibitory concentration) and MBC (e.g., minimum bactericidal concentration) were used. 96 well micro-well plates were placed in a humid chamber and frozen at −70° C. [BBL Mueller Hinton Broth]. Drugs (e.g., antimicrobial compounds of the present invention) were added to a series of wells through 2 fold serial dilutions. 100 μl of a 2×10⁻³ M solution of the respective Tx compounds was added to the first well in a series. 100 μl of the inoculum (e.g., microorganism) was added to the well. Thus, the final concentration of a Tx compound in well 1 was 1×10⁻³ M. The concentration of drug in each successive well in the series from 1-10 had a lower concentration than the previous well in the series. For example, the concentration in well 8 would be less than the concentration of the same drug in well 7. Inoculum (e.g., microorganism) that was added to the wells was prepared from 24 hour growth of an isolate on 5% sheep blood/Columbia agar plates. 5 to 10 representative colonies were then picked from the plates and added to saline and adjusted to 0.5 McFarland turbidity. Next the sample was diluted 1:100 in saline, such that 100 μl of the diluted sample contained approximately 1×10⁵ microbes. 100 μl of the diluted sample was added to each well in a series of drug concentrations. Once the cells were added to the wells, the well plates were incubated at 35° C. without CO₂ for 48 hrs and at this time the minimum bactericidal concentration was determined. MIC of a drug for a particular organism was found to be relatively stable, when analyzed at 48 and 24 hours. The results of testing for various microorganisms are summarized in Table 2.

TABLE 2

| Microbe Identification | | MIC/MBC* ug/ml | | | |
|---|---|---|---|---|---|
| Plate | Microbe | Tx-5 | Tx-147 | Tx-37 | Tx-38 |
| A1 | MRSA | 3/3 | 24 | 7.2 | +++^{a} |
| B2 | MRSA | 1.5 | 48 | 3.6 | +++ |
| C3 | S. maltophilia | 48/48 | 96 | +++ | +++ |
| D4 | VRE | 3 | 12 | 58 | +++ |
| E5 | MRSA | 1.5 | 24 | 3.6 | +++ |
| F6 | MRSA | 1.5/1.5 | 24 | 1.8 | +++ |
| G7 | MRSA | 1.5 | 24 | 3.6 | +++ |
| H9 | MRSA | 3/3 | 48 | 7.2 | +++ |
| I10 | S. maltophilia | 24/24 | 784 | +++ | +++ |
| J11 | S. maltophilia | 48/48 | 784 | +++ | +++ |
| K12 | MRSA | 3 | 48 | 7.2 | +++ |
| L13 | P. aeruginosaR | +++ | +++ | +++ | +++ |
| ATCC | P. aeruginosa | +++ | +++ | +++ | +++ |
| 37 | P. aeruginosa | +++ | +++ | +++ | +++ |
| EC | E. coli | 48/48 | +++ | +++ | +++ |
| E502 | Enterococcus | 1.5/6 | 6/12 | +++ | +++ |
| E348 | Enterococcus | 1.5/3 | 3/6 | +++ | +++ |
| E523 | Enterococcus | 1.5/1.5 | 6/12 | +++ | +++ |
| S332 | S. aureus | 0.4/0.7 | 12/12 | +++ | +++ |
| S520 | S. aureus | 1.5/1.5 | 12/12 | +++ | +++ |
| 699 | VancoR S. aureus^{&} | 1.5/12 | 6/23 | NT | NT |
| 788 | VancoR S. aureus^{&} | 3/12 | 24/49 | NT | NT |

All except ATCC strains are patient isolates derived from M. D. Anderson Cancer Center
*= Minimum bactericidal concentration was read at 48 hr. and indicates 10⁻³ kill.
^{a}= +++ indicates growth in all wells (no activity).
VRE = Vancomycin resistant Enterococci (all are also resistant to ampicillin).
MRSA = Oxacillin resistant S. areus. (all are resistant to penicillin and Cefzox)
S. maltophilia are only sensitive to trimethoprim/sulfa.

All except ATCC strains are patient isolates derived from M.D. Anderson Cancer Center.

*=Minimum bactericidal concentration was read at 48hr and indicates 10⁻³ kill.

^{a}=+++ indicates growth in all wells (no activity).

VRE=Vancomycin resistant Enterococci (all are also resistant to ampicillin).

MRSA=Oxacillin resistant S. aureus. (all are resistant to penicillin and Cefzox) S. maltophilia are only sensitive to trimethoprim/sulfa.

A1=sensitive to clindamycin, gentamycin, rifampin, sulfamethoazole/trimethoprim, and vancomycin. A1 resistant to amoxicillin/clavulanate, amoxicillin/sulfamethoxalone, ciprofloxacin, erythromycin, ofloxacin, penicillin, and tetracyclin.

B2=sensitive to clindamycin, gentamycin, rifampicin, sulfamethoazole/trimethoprim, and vancomycin. A1 resistant to amoxicillin/clavulanate, amoxicillin/sulfamethoxalone, ciprofloxacin, erythromycin, ofloxacin, penicillin, and tetracyclin.

E5=sensitive to clindamycin, tetracycline, gentamycin, rifampicin, sulfamethoazole/trimethoprim, and vancomycin. A1 resistant to amoxicillin/clavulanate, amoxicillin/sulfamethoxalone, erythromycin, ofloxacin, and penicillin.

F6=sensitive to clindamycin, gentamycin, rifampicin, sulfamethoazole/trimethoprim, and vancomycin. A1 resistant to amoxicillin/clavulanate, amoxicillin/sulfamethoxalone, ciprofloxacin, erythromycin, ofloxacin, penicillin, and tetracyclin.

G8=sensitive to clindamycin, tetracycline, gentamycin, rifampicin, sulfamethoazole/trimethoprim, and vancomycin. A1 resistant to amoxicillin/clavulanate, amoxicillin/sulfamethoxalone, erythromycin, ofloxacin, and penicillin.

H9=sensitive to clindamycin, tetracycline, gentamycin, rifampicin, sulfamethoazole/trimethoprim, and vancomycin. A1 resistant to amoxicillin/clavulanate, amoxicillin/sulfamethoxalone, erythromycin, ofloxacin, and penicillin.

I10=S. maltophilia sensitive to ceftzidime, ticarcillin/clavulanate, sulfamethoxazole/trimethoprim and resistant to amikacin and ciprofloxacin.

J11=S. maltophilia sensitive to ticarcillin/clavulanate and sulfamethoxazole/trimethoprim and resistant to ceftzidime, amikacin and ciprofloxacin.

L13=P. aeruginosaR is resistant to aztreonam, cefepime, ceftazidime, cefzox, ceftriaxone, ciprofloxacin, imipenem, levofloxacin, meropenem, norfloxacin, piperacillin, piperacillin/tazobactam, ticarcillin/clavulanate.

&=Vancomycin resistant S. aureus ATCC700699 and 700788.

NT=Not tested.

37=P. aeruginosa sensitive to amikacin, aztreonam, cefepime, ceftazidime, ceftriaxone, ciprofloxacin, gentamicin, imipenem, levofloxacin, meropenem, piperacillin, piperazillin/tazobactam, ticarcillin/clavulanate, tobramycin.

ATCC=P. aeruginosa ATCC 27853 sensitive to amikacin, cefoxitin, ceftrzoxime, cipromycin, levofloxacin, meropenem, norfloxacin, ofloxacin, resistant to amoxicillin/clavulanate, cefotetan, cefoxitin, ceftazidime, cephalothin, tetracycline, ticarcillin, ticarcillin/clavulanate.

EC=E. coli ATCC 25922 is pan sensitive=amikacin, amoxicillin/clavulanate, cefotaxime, cefotetan, cefoxitin, cefpodoxime, ceftrzoxime, cephalothin, meropenem, norfloxacin, ofloxacin, tetracycline, ticarcillin, ticarcillin/clavulanate.

E502=Enterococcus faecium ATCC 502806 is sensitive to penicillin, gentamycin, streptomycin and vancomycin.

E348=Enterococcus faecium patient isolate is sensitive to cipromycin, gentamycin, levofloxacin, penicillin, streptomycin, tetracycline, and vancomycin.

E523=Enterococcus facium patient isolate sensitive to ciprofloxacin, gentamycin, levofloxacin, penicillin, and vancomycin, and tetracycline resistant.

S332=S aureus patient isolate resistant to penicillin, ampicillin, oxacillin, amoxicillin/clavulanate, amoxicillin/sulfamethoxalone, cefoxitin, clindamycin, and erythromycin.

S520=S aureus patient isolate penicillin resistant.

All of the S. maltophilias were trimethoprim/sulfamethoxalone sensitive.

Tx-5 and Tx-147 showed bactericidal activity against Staphylococcus aureus (MRSA). Tx-5 also showed some bactericidal activity against Stenotrophomonas maltophilia. Tx-147 was found to have some bactericidal activity for VRE. Compounds Tx-9, Tx-10, Tx-11, and Tx-12 all showed +3 growth (e.g., no activity). The results of Table 2 were converted to μg/ml and are provided in Table 3 based on molecular weights of drugs, along with results for M. tuberculosis, M. avium intracellularie, M. chelonae, C. albicans, A. fumigatus, and Fusarium. Results for toxicity studies with human fibroblasts and animal studies are also in Table 3. To determine the toxicity of these compounds in vivo they were tested in the mouse strain BDF_{1′} mice of both sexes. The respective agents were diluted in tissue culture grade DMSO and administered intraperitoneally in a volume not to exceed 5 microliters. The testing included single doses, doses given on alternate days and doses given daily 5 days a week. In some instances the daily or alternate day regiment were repeated for a period of one month.

The molecular weights of Tx-1, Tx-2, Tx-3, Tx-4,Tx-5, Tx-8, Tx-118, Tx-147, Tx-197, Tx-37, and Tx-38 are 426, 426, 411, 411, 382, 536, 431, 785, 389, 463, and 448, respectively.

TABLE 3
MIC (24 h and 48 h same results) for following compounds and toxicity studies:

| | TX118 | TX147* | TX197 |
|---|---|---|---|
| MRSA (methicillin resistant) | No activity | 24-49 ug/ml | No activity |
| VRSA (ATCC 700699) | | 6 ug/ml | |
| VRSA (ATCC 700788) | | 24 ug/ml | |
| P. aeruginosa | No activity | No activity | No activity |
| Stenotrophomonas | No activity | 98-785 ug/ml, 78 ug/ml (resist. | No activity |
| maltophilia | | various drugs) | |
| VRE enterococcus | No activity | 12 ug/ml, 19 ug/ml (vancomycin, | No activity |
| (E. faecalis) | | Penicillin & aminoglycoside rest. | |
| Mycobacterium fortutium | No activity | 39 ug/ml | No activity |
| Mycobacterium tuberculosis | | 3 ug/ml | |
| M. avium intracellularie | | 12-24 ug/ml | |
| *animal studies (ip) | | 30 mg/kg (10-12% mort) | |

| | TX1 | TX2 | TX3 | TX4 | TX5* | |
|---|---|---|---|---|---|---|
| MRSA | | | | | 1.5-3 | ug/ml |
| VRSA (ATCC 700699) | | | | | 1.5 | ug/ml |
| VRSA (ATCC 700788) | | | | | 3.0 | ug/ml |
| P. aeruginosa | | | | | No activity | |
| Stenotrophomonas | | | | | 24-48 | ug/ml |
| maltophilia | | | | | | |
| VRE enterococcus | | | | | 3 | ug/ml |
| (E. faecalis) | | | | | | |
| Mycobacterium fortutium | | | | | | |
| Mycobacterium chelonae | | | | | 5-24 | ug/ml |
| Mycobacterium tuberculosis | 2.9 ug/ml | 1 ug/ml | .125 ug/ml | .1 ug/ml | 7 | ug/ml |
| **M. tuberculosis resisant. strains | | | | | 2 | ug/ml 2 wk; |
| | | | | | 4 | ug/ml 4 wk |
| M. avium intracellularie | 46 ug/ml | 15.6 ug/ml | .98 ug/ml | .82 ug/ml | 4, 7.5, 15 | ug/ml(3 exp.) |
| Candida albicans | | | | | 30 | ug/ml; |

| | (90% inhib. @ 15 ug/ml) | |
|---|---|---|
| Toxicity/human fibroblasts (IC50) | 6 | ug/ml |
| *animal studies (ip) | 35 | mg/kg (<10% mort) |

| | TX37* | TX38* | TX84 | TX112 |
|---|---|---|---|---|
| MRSA | 1.8-7 ug/ml | No activity | 16 ug/ml | |
| VRSA (ATCC 700699) | | | | 8 ug/ml |
| VRSA (ATCC 700788) | | | | 8 ug/ml |
| P. aeruginosa | No activity | No activity | No activity | No activity |
| Stenotrophomonas | No activity | No activity | | |
| maltophilia | | | | |
| VRE enterococcus | 58 ug/ml | No activity | 4-33 ug/ml | 210 ug/ml |
| (E. faecalis) | | | | |
| Mycobacterium fortutium | | | | |
| Mycobacterium tuberculosis | | | | |
| M. avium intracellularie | | | | |
| Candida albicans | | | | |
| Aspergillus fumigatus | | | | 6.5 ug/ml |
| Fusarium | | | | 3.3 ug/ml |

| | | |
|---|---|---|
| Toxicity/human fibroblasts (IC50) | 5 ug/ml | 12 ug/ml human fibroblasts |
| | | (IC50) |
| *animal studies (ip) | 30 mg/kg (no mortality) | 30 mg/kg (no mort) |

TX9, 10, 11, 12 had no activity against Mycobacterium tuberculosis, M. avium intracelluarie, or C. albicans up to 30 ug/ml. Tx-225 has activity similar to Tx-84, but is expected to be more soluble. P. aeroginosa is L13 from Table 2, above.

### EXAMPLE 2

The MIC of Tx-118, Tx-147, and Tx-197 for certain Staphylococcus aureus (MRSA), Pseudomonas aeruginosa, Stenotrophomonas maltophilia, and VRE Enterococcus faecalis was determined using standard NCCLS methods. The first well in each series of decreasing drug concentrations contained 1×10⁻⁴ M of a drug. The drug was diluted by serial two fold dilutions through all 12 wells in a series. There was a final DMSO concentration of 2% in the first well and 1% in the second well, etc. Tests with Tx-118 and Tx-197 were performed in triplicate, and tests with Tx-147 were performed in duplicate. Based on this experiment, compound Tx-147 was active against a broad range of resistant organisms, and was bactericidal at certain concentrations for the Staphylococcus aureus (MRSA) that was tested. Tx-147 has a molecular weight of 784, and the concentration of the drug in wells 1 and 2 that was bactericidal was 78×10⁻⁴ μg/ml and 39×10⁻⁴ μg/ml. The third and fourth wells corresponded to a concentration for Tx-147 of 19×10⁻⁴ μg/ml and 9.7×10⁻⁴ μg/ml. The MIC of Tx-147 for the Staphylococcus aureus tested was 39×10⁻⁴μg/ml. The MIC of Tx-147 for the Stenotrophomonas maltophilia tested was 78×10⁻⁴ μg/ml. The MIC of Tx-147 for the VRE tested was 19×10⁻⁴ μg/ml, and 39×10⁻⁴ μg/ml for the tested Mycobacterium fortutium (see above).

### EXAMPLE 3

Tx-5 and Tx-84 were tested for in vitro efficacy against a chloroquin (CQ) resistant strain of Plasmodium falciparum (Pf). The tests were performed in triplicate at a wide variety of concentrations and evaluated for effect compared with CQ. The results are demonstrated in Table 4, and Tx-84 had a significant inhibitory effect, while Tx-5 had a weak effect. Tx-84 inhibitory effectiveness was greater than that of CQ against Pf.

TABLE 4

| Compound | IC₅₀ (M) |
|---|---|
| Tx-5 | 1.54 × 10⁻⁴ |
| Tx-84 | 9.1 × 10⁻⁸ |
| CQ | 6.06 × 10⁻⁷ |

The preceding description of specific embodiments of the present invention is not intended to be a complete list of every possible embodiment of the invention. Persons skilled in this field will recognize that modifications can be made to the specific embodiments described here that would be within the scope of the present invention.

1. A method of inhibiting the growth of at least one microorganism, comprising the step of administering to an animal in an amount effective to inhibit microbial growth at least one compound having a formula selected from the group consisting of 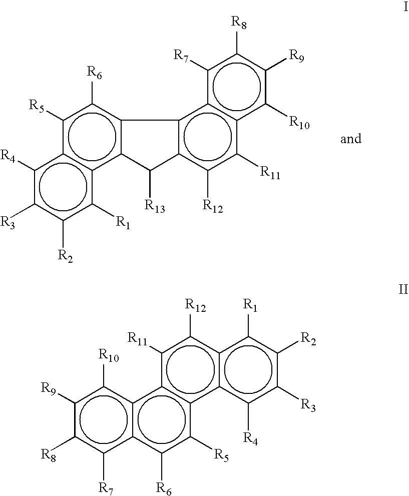
or a salt thereof;
where at least one of R₁-R₁₃ in formula (I) or at least one of R₁-R₁₂ in formula (II) is —R₁₄Z, where R₁₄ is a substituted or unsubstituted linking group comprising from 1-12 carbon atoms, and Z is a substituted or unsubstituted heterocyclic group having from 1-12 carbon atoms;
where the remainder of R₁-R₁₃ in formula (I) are independently selected from the group consisting of hydrogen, hydroxyl, halogen, nitro, methoxy, acyl, alkyl groups having from 1-12 carbon atoms, and substituted or unsubstituted pendant groups comprising (i) from 1-12 carbon atoms and (ii) at least one of an amino group or an amido group; and
where the remainder of R₁-R₁₂ in formula (II) are independently selected from the group consisting of hydrogen, nitro, substituted or unsubstituted hydrocarbyl groups having from 1-12 carbon atoms, and substituted or unsubstituted pendant groups comprising (A) from 1-12 carbon atoms and (B) at least one of an amino group or an amido group.

2. The method of claim 1, wherein the R₁₄ comprises at least one of an amino or an amido group.

3. The method of claim 1, where R₁₁ of formula (I) is —R₁₄Z.

4. The method of claim 1, where R₁₄ has the formula —NHR₁₅—, where R₁₅ is a substituted or unsubstituted aliphatic group having from 2-6 carbon atoms.

5. The method of claim 4, where R₁₅ is selected from the group consisting of —CO(CH₂)ₙCO— and —(CH₂)ₘ— where n is from 1-4, and m is from 2-6.

6. The method of claim 1, where Z is selected from the group consisting of morpholinyl, pyrrolidinyl, piperidinyl and piperazinyl.

7. The method of claim 1, where R₂ of formula (II) is —R₁₄Z and R₁ and R₃-R₁₂ are hydrogen.

8. The method of claim 1, where R₆ of formula (II) is —R₁₄Z and R₁-R₅ and R₇-R₁₂ are hydrogen.

9. The method of claim 1, wherein both R₆ and R₁₂ of formula (II) are —R₁₄Z and R₁-R₅, and R₇-R₁₁ are hydrogen.

10. The method of claim 1, wherein the microorganism belongs to a genus selected from the group consisting of Staphylococcus, Stenotrophomonas, Enterococcus, Mycobacterium, Plasmodium, Pseudomonas, and Candida.

11. The method of claim 1, wherein the microorganism is selected from the group consisting of Staphylococcus aureus, Stenotrophomonas maltophilia, vancomycin-resistant Enterococcus faecium (VRE), Mycobacterium fortutium, Mycobacterium tuberculosis, Mycobacterium avium intracellulare, Plasmodium falciparum, Pseudomonas aeruginosa, and Candida albicans.

12. The method of claim 1, where the compound has formula (I) and is
N-[11′-(13′H-Dibenzo[a,g]-fluorenyl)]-4-(4′N methyl-piperazinyl)-butane-1,4-dicarboxiamide.

13. The method of claim 12, wherein the microorganism is Staphylococcus aureus or vancomycin-resistant Enterococcus faecium (VRE).

14. The method of claim 1, wherein the method comprises co-administering at least one additional antimicrobial compound.

15. The method of claim 1, where the compound has formula (II) and is selected from the group consisting of
N-[(6′-chrysenyl)-4-(4′N methyl-piperazinyl)-butane-1,4-diamine]-N-[(12′ chrysenyl)-4-(4′N methyl-piperazinyl)-butane-1,4-diamine hydrochloride;
N-[(6′-chrysenyl)-4-piperidinyl-butane-1,4-diamine]-N-[(12′ chrysenyl)-4-piperidinyl-butane-1,4-diamine;
N-[(6′-chrysenyl)-4-piperidinyl-butane-1,4-diamine]-N-[(12′ chrysenyl)-4-piperidinyl-butane-1,4-diamine hydrochloride;
N-(2′-chrysenyl)4-(1′-piperidinyl)-butane-1,4-diamine;
N-[(6′-chrysenyl)-4-(4′N methyl-piperazinyl)-butane-1,4-diamine]-N-[(12′ chrysenyl)-4-(4′N methyl-piperazinyl)-butane-1,4-diamine;
N-(6′-chrysenyl)-4-(4′N methyl-piperazinyl)-butane-1,4-dicarboxiamide;
N-(2′-chrysenyl)-4-(4′N-methyl-piperazinyl)-butane-1,4-dicarboxiamide;
N-(6′-chrysenyl)-4-(1′ piperidinyl)-butane-1,4-dicarboxiamide;
N-(2′-chrysenyl)-4-(1′piperidinyl)-butane-1,4-dicarboxiamide; and
N-(2′-chrysenyl)4-( 1′-piperidinyl)-butane-1,4-diamine.

16. The method of claim 1, where the compound has formula (II) and is selected from the group consisting of
N-(6′-chrysenyl)-4-(4′N methyl-piperazinyl)-butane-1,4-dicarboxiamide;
N-(2′-chrysenyl)-4-(4′N-methyl-piperazinyl)-butane-1,4-dicarboxiamide;
N-(6′-chrysenyl)-4-(1′ piperidinyl)-butane-1,4-dicarboxiamide;
N-(2′-chrysenyl)-4-(1′ piperidinyl)-butane-1,4-dicarboxiamide; and
N-(2′-chrysenyl)4-(1′-piperidinyl)-butane-1,4-diamine.

17. The method of claim 16, wherein the microorganism is Mycobacterium tuberculosis or Mycobacterium avium intracellulare.

18. The method of claim 1, where the compound has formula (II) and is N-(2′-chrysenyl)-4-(1′-piperidinyl)-butane-1,4-diamine.

19. The method of claim 18, wherein the microorganism is selected from the group consisting of Stenotrophomonas maltophilia, vancomycin-resistant Enterococcus faecium (VRE), Candida albicans, and Staphylococcus aureus.

20. The method of claim 1, where the compound has formula (II) and is
N-[(6′-chrysenyl)-4-(4′N methyl-piperazinyl)-butane-1,4-diamine]-N-[(12′ chrysenyl)-4-(4′N methyl-piperazinyl)-butane-1,4-diamine.

21. The method of claim 20, wherein the microorganism is selected from the group consisting of Staphylococcus aureus, Stenotrophomonas maltophilia, vancomycin-resistant Enterococcus faecium (VRE), and Mycobacterium fortutium.

22. A method of inhibiting the growth of at least one microorganism, comprising the step of administering to an animal in an amount effective to inhibit microbial growth of at least one compound having the formula (A), 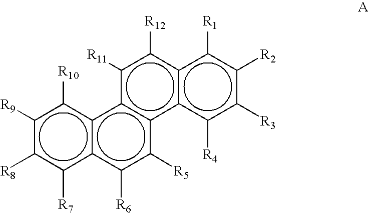
or a salt thereof;
where at least one of R₁-R₁₂ is —R₁₄Z, where R₁₄ is a substituted or unsubstituted linking group having from 1-12 carbon atoms and (b) at least one of an amino group or amido group, and Z is a piperazinyl or a piperidinyl group; and
where the remainder of R₁-R₁₂ are independently selected from the group consisting of hydrogen, nitro, substituted or unsubstituted hydrocarbyl groups having from 1-12 carbon atoms, and substituted or unsubstituted pendant groups having from 1-12 carbon atoms and at least one of an amido group or an amino group,.

23. The method of claim 22, where R₂is —R₁₄Z and R₁ and R₃-R₁₂ are hydrogen.

24. The method of claim 22, where R₆ is —R₁₄Z and R₁-R₅ and R₇-R₁₂ are hydrogen.

25. The method of claim 22, where R₁₄ has the formula —NHR₁₅—, where R₁₅ is a substituted or unsubstituted aliphatic group having from 2-6 carbon atoms.

26. The method of claim 25, where R₁₅ is selected from the group consisting of —CO(CH₂)ₙCO—, and —(CH₂)ₘ—, where n is from 1-4, and m is from 2-6.

27. The method of claim 22, wherein the microorganism is selected from the group consisting of Staphylococcus aureus, Stenotrophomonas maltophilia, vancomycin-resistant Enterococcus faecium (VRE), Mycobacterium fortutium, Mycobacterium tuberculosis, Mycobacterium avium intracellulare, Pseudomonas aeruginosa, Plasmodium falciparum and Candida albicans.

28. The method of claim 22, where the compound is selected from the group consisting of:
N-[(6′-chrysenyl)-4-(4′N methyl-piperazinyl)-butane-1,4-diamine]-N-[(12′ chrysenyl)-4-(4′N methyl-piperazinyl)-butane-1,4-diamine hydrochloride;
N-[(6′-chrysenyl)-4-piperidinyl-butane-1,4-diamine]-N-[(12′ chrysenyl)-4-piperidinyl-butane-1,4-diamine;
N-(2′-chrysenyl)4-(1′-piperidinyl)-butane-1,4-diamine;
N-[(6′-chrysenyl)-4-(4′N methyl-piperazinyl)-butane-1,4-diamine]-N-[(12′ chrysenyl)-4-(4′N methyl-piperazinyl)-butane-1,4-diamine;
N-(6′-chrysenyl)-4-(4′N methyl-piperazinyl)-butane-1,4-dicarboxiamide;
N-(2′-chrysenyl)-4-(4′N-methyl-piperazinyl)-butane-1,4-dicarboxiamide;
N-(6′-chrysenyl)-4-(1′ piperidinyl)-butane-1,4-dicarboxiamide;
N-(2′-chrysenyl)-4-(1′ piperidinyl)-butane-1,4-dicarboxiamide; and
N-(2′-chrysenyl)4-(1′-piperidinyl)-butane-1,4-diamine.

29. The method of claim 22, where the compound is selected from the group consisting of
N-(6′-chrysenyl)-4-(4′N methyl-piperazinyl)-butane-1,4-dicarboxiamide;
N-(2′-chrysenyl)-4-(4′N-methyl-piperazinyl)-butane-1,4-dicarboxiamide;
N-(6′-chrysenyl)-4-(1′ piperidinyl)-butane-1,4-dicarboxiamide;
N-(2′-chrysenyl)-4-(1′ piperidinyl)-butane-1,4-dicarboxiamide; and
N-(2′-chrysenyl)-4-(1′-piperidinyl)-butane-1,4-diamine.

30. The method of claim 29, wherein the microorganism is Mycobacterium tuberculosis, Plasmodium falciparum, or Mycobacterium avium intracellulare.

31. The method of claim 22, where the compound is N-(2′-chrysenyl)4-(1′-piperidinyl)-butane-1,4-diamine.

32. The method of claim 31, wherein the microorganism is selected from the group consisting of Stenotrophomonas maltophilia, vancomycin-resistant Enterococcus faecium (VRE), Candida albicans, Plasmodium falciparum, and Staphylococcus aureus.

33. The method of claim 22, where the compound is
N-[(6′-chrysenyl)-4-(4′N methyl-piperazinyl)-butane-1,4-diamine]-N-[(12′ chrysenyl)-4-(4′N methyl-piperazinyl)-butane-1,4-diamine.

34. The method of claim 33, wherein the microorganism is selected from the group consisting of Staphylococcus aureus, Stenotrophomonas maltophilia, vancomycin-resistant Enterococcus faecium (VRE), and Mycobacterium fortutium.

35. The method of claim 22, wherein the method comprises co-administering at least one additional antimicrobial compound.

36. A method of inhibiting the growth of at least one microorganism, comprising the step of administering to an animal in an amount effective to inhibit microbial growth at least one compound having the formula B, 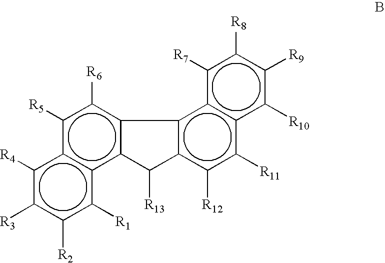
or a salt thereof,
where R₁-R₁₀, R₁₂, and R₁₃ are independently selected from the group consisting of hydrogen, hydroxyl, halogen, nitro, methoxy, acyl, alkyl groups having from 1-12 carbon atoms, and substituted or unsubstituted chemical groups comprising (i) from 1-12 carbon atoms and (ii) at least one amino or amido group;
where R₁₁ is —R₁₄Z;
where R₁₄ has the formula —NHR₁₅—, where R₁₅ is a substituted or unsubstituted aliphatic group having from 2-6 carbon atoms; and
where Z is a piperazinyl or a piperidinyl group.

37. The method of claim 36, where R₁₅ is selected from the group consisting of —CO(CH₂)ₙCO— and —(CH₂)ₘ— where n is from 1-4, and m is from 2-6.

38. The method of claim 36, wherein the microorganism is Staphylococcus aureus, or vancomycin-resistant Enterococcus faecium (VRE).

39. The method of claim 36, where the compound is
N-[11′-(13′H-Dibenzo[a,g]-fluorenyl)]-4-(4′N methyl-piperazinyl)-butane-1,4-dicarboxiamide.

40. The method of claim 39, wherein the microorganism is Staphylococcus aureus, or vancomycin-resistant Enterococcus faecium (VRE).

41. The method of claim 36, wherein the method comprises co-administering at least one additional antimicrobial compound.

42. A method of prophylaxis or treatment of a viral infection in an animal, comprising administering to an animal at least one compound having a formula 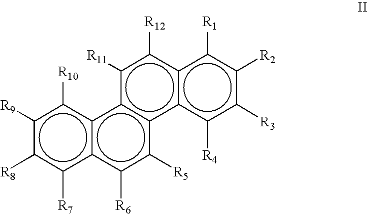
or a salt thereof;
where at least one of R₁-R₁₂ in formula (II) is —R₁₄Z, where R₁₄ is a substituted or unsubstituted linking group comprising from 1-12 carbon atoms, and Z is a substituted or unsubstituted heterocyclic group having from 1-12 carbon atoms;
where the remainder of R₁-R₁₂ in formula (II) are independently selected from the group consisting of hydrogen, nitro, substituted or unsubstituted hydrocarbyl groups having from 1-12 carbon atoms, and substituted or unsubstituted pendant groups comprising (A) from 1-12 carbon atoms and (B) at least one of an amino group or an amido group;
wherein the at least one compound having formula (II) or salts thereof is administered in an amount effective to inhibit infection of at least some of the animal's cells by at least one virus.

43. The method of claim 42, wherein the method comprises coadministering an additional antiviral compound.

44. A method of prophylaxis or treatment of a viral infection in an animal, comprising administering to an animal at least one compound having a formula 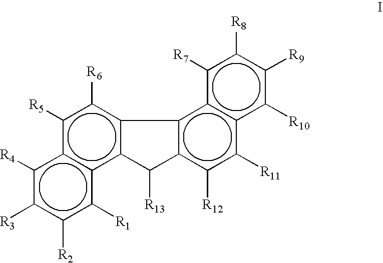
or a salt thereof;
where at least one of R₁-R₁₃ in formula (I) is —R₁₄Z, where R₁₄ is a substituted or unsubstituted linking group comprising from 1-12 carbon atoms, and Z is a substituted or unsubstituted heterocyclic group having from 1-12 carbon atoms;
where the remainder of R₁-R₁₃ in formula (I) are independently selected from the group consisting of hydrogen, hydroxyl, halogen, nitro, methoxy, acyl, alkyl groups having from 1-12 carbon atoms, and substituted or unsubstituted pendant groups comprising (i) from 1-12 carbon atoms and (ii) at least one of an amino group or an amido group; and
wherein the at least one compound having formula (I) or salts thereof is administered in an amount effective to inhibit infection of at least some of the animal's cells by at least one virus.

45. The method of claim 44, wherein the method comprises coadministering an additional antiviral compound.

46. N-[(6′-chrysenyl)-4-(4′N methyl-piperazinyl)-butane-1,4-diamine]-N-[(12′ chrysenyl)-4-(4′N methyl-piperazinyl)-butane-1,4-diamine hydrochloride.

47. Trans-1-N-(6′-chrysenyl)-3-hydroxy-4-phenyl-2-azetidinone.

48. N-[(6′chrysenyl)-4-piperidinyl-butane-1,4-diamine]-N-[(12′chrysenyl)-4-piperidinyl-butane-1,4-daimine hydrochloride.
